(19) United States
(12) Patent Application Publication
(10) Pub. No.: US 2025/0359523 A1
(43) Pub. Date: Nov. 27, 2025

(54) POLLEN DISTRIBUTION DEVICE FOR CROP PLANTS IN AN AGRICULTURAL FIELD, CORRESPONDING USE AND OPERATING METHOD

(71) Applicant: BASF SE, Ludwigshafen am Rhein (DE)

(72) Inventors: Wilmar Ernesto MORJAN, Ecully Cedex (FR); Stephane POUZADOUX, Ecully Cedex (FR); Tyfanie BOURLET, Ecully Cedex (FR)

(21) Appl. No.: 18/867,664

(22) PCT Filed: Apr. 28, 2023
(86) PCT No.: PCT/EP2023/061351

§ 371 Date: Nov. 20, 2024

(30) Foreign Application Priority Data
May 20, 2022 (EP) 22174691.0

(51) Int. Cl. A01H 1/02

(52) U.S. Cl. CPC A01H 1/027

(57) ABSTRACT

The invention relates to a pollen distribution device for crop plants in an agricultural field, the device comprising a carrier structure configured to be moved along rows of crop plants of the agricultural field in a moving direction, a blowing device attached to the carrier structure, wherein the blowing device comprises at least one nozzle arrangement configured to blow air against pollen bearing plants of the agricultural field. According to the invention it is proposed that the nozzle arrangement comprises a vaporizing device for adding humidity to the air leaving the nozzle arrangement, in particular wherein the vaporizing device is configured to increase the relative humidity of the air leaving the nozzle arrangement between 60% and 80% to ensure high relative humidity suitable for pollen viability preservation by atomizing the water particles.

The invention relates to a pollen distribution device for crop plants in an agricultural field, the device comprising a carrier structure configured to be moved along rows of crop plants of the agricultural field in a moving direction and a blowing device attached to the carrier structure, wherein the blowing device comprises at least one nozzle arrangement configured to blow air against pollen bearing plants of the agricultural field.

There are several systems available in different crops (such as cereal crops, but also others) to obtain male sterility so as to produce hybrid seed on female plants by cross-pollination. Some of these systems include cytoplasmic male sterility (“CMS”), chemically-induced male sterility (using a Chemical Hybridizing Agent (CHA)), or nuclear male sterility (“NMS”). In any of these systems, efficient pollination of (male-sterile) female plants by (pollen-producing) male plants is required, and ideally no or little male seed ends up in harvested hybrid seed.

There are essentially 2 methods to obtain cross-pollination between male and female plants:mixed planting, where the male (pollen-donating) plants are randomly or uniformly distributed amongst female (male-sterile) plants at some percentage, and strip (or row or bay) planting, where the male (pollen-donating) plants are physically separated from the female (male-sterile) plants in separate rows/field parts.

In a mixed planting approach, the male (pollen-donating) plants are randomly or uniformly distributed amongst female (male-sterile) plants at some percentage, but as the male plants self-pollinate, male seed will also be produced in this setup, and that needs to be removed to obtain hybrid seed of sufficient purity (to meet requirements of customers or (certification) authorities), or can only be present in limited quantity in the female field, which reduces the amount of pollen-providing males.

Self pollinated males are most easily removed in strip planting (also known as bay planting) or row planting system where one or more female rows are planted next to one or more male rows, by removing the male plants that are physically separated from the female rows (after pollination of the female plants), or by ensuring that the male plants do not produce (normal size) seed, so that only the hybrid seed produced on the female plants is harvested (or smaller male seed is removed at harvest). It may also be ensured that the male rows are excluded during harvest.

A key concern in the success of hybrid row crops like wheat, rice or corn is the economics of seed production, which has to be efficient and cost-effective (Whitford et al., 2013, Gupta et al., 2019), mainly due to the tendency to self-pollinate in these crops. Also, some row crops rely on pollination by insects and in Europe, a dramatic decline in the occurrence and diversity of all kinds of wild insect pollinators has been observed (like wild bees, hoverflies, butterflies and moths) (see, e.g., https://ec.europa.eu/environment/nature/conservation/species/pollinators/index_en.htm), so that pollination efficiency decreases.

WO2019/175507 and WO2022/023663 disclose an air-moving device employing the Coanda effect, that collects pollen on pollen donor plants, which pollen is moved through the device, and distributes the pollen collected on recipient plants. It is said to be particularly useful for crops with recalcitrant pollen that does not stay viable for long (such as wheat, barley or corn).

WO2018/129302 discloses a device for pollinating plants, containing a pollination unit mounted on a base mountable on a carriage, including a pollen-releasing apparatus configured to release pollen from male flowers of the plants and at least one nozzle for directing at least some of the released pollen to the pollen-receiving rows. Plants are pollinated by driving the carriage along a field and directing air from the nozzles over the pollen born by some plants to deliver the pollen to female flowers of other plants in the field.

The article “Pollen viability in the field” by M. Bots and C. Mariani relates to a review of scientific literature on pollen viability and to analysing pollen viability in the field. However, the document does not discuss or disclose to influence environmental conditions by adding humidity actively in order to provide conditions for pollen preservation.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

However, there remains a need for a simple and effective improved system of plant cross-pollination, particularly for row crops such as but not limited to cereal crops (like wheat, rice or corn), both for female parent maintenance, or hybrid seed production. Thus, it was an object of the invention to provide an improved pollen distribution device that improves the overall plant cross-pollination and preserves pollen viability.

The invention attains the aforementioned object by suggesting a pollen distribution device according to claim 1. According to one embodiment of the invention, the pollen distribution device comprises at least one nozzle arrangement comprising a vaporizing device for adding humidity to the air leaving the nozzle arrangement, in particular wherein the vaporizing device is configured to increase the relative humidity of the air from the nozzle arrangement to ensure high relative humidity suitable for pollen viability preservation by atomizing the water particles (such between 60% and 80% humidity). The relative humidity may, for example, be (at least) 60%, 65%, 70%, 75% or 80% or in the range of 60%-70% or 70%-80%. The vaporizing device may be configured as an atomizing device configured for atomizing a liquid such as water that is provided to the atomizing device. The vaporizing device is utilized to atomize the water in order to humidify the air leaving the nozzle arrangement (utilizing pressure and atomizing nozzles to create an aerosol or small droplets suspended in air).

This embodiment of the invention achieves the following advantages: The proposed device improves pollen distribution from the male plants to female plants. By increasing humidity in the air being blown to the pollen, dehydration of the pollen is reduced, and temperature of the air flow is decreased further improving viability of the pollen. Adjusting or increasing the relative humidity to between 60% and 80% has been found to be beneficial for pollen viability preservation. Said relative humidity is however low enough to avoid the pollen from absorbing too much humidity and getting wet which would significantly reduce the travelling distance of the pollen. Furthermore, the device is also suitable for use with row crop plants having pollen that can partially dehydrate and rehydrate but that results in lower amounts of viable pollen, or for pollination of plants during warm temperatures, such as above 25° C. or 30° C.

According to one embodiment, an exhaust direction of the air leaving the nozzle arrangement is inclined upwards with respect to a vertical direction by an angle of 1° to 45°, preferably 100 in order to suspend the pollen upwards. By suspending the pollen upwards, the travelling distance of the pollen can be increased. The angle may be in the range of 1° to 15°, 15° to 30°, 30° to 45° or have individual values like 10°, 15°, 20°, 25°, 30°, 35°, 40°. The expression “suspending pollen” is to be understood herein as to blow or generally move the pollen.

Preferably, an exhaust direction of the air leaving the nozzle arrangement is tilted rightwards or leftwards with respect to a longitudinal axis of the device, in particular outwards with respect to the longitudinal axis. In other words, by tilting the exhaust direction of the air leaving the nozzle arrangement outwards, the air is directed towards the rows of female (pollen-receiving) crop plants. According to another embodiment, the device comprises a blower, in particular a centrifugal blower, the blower being fluidly connected to the nozzle arrangement. The blower may be arranged on the device itself. According to an alternative embodiment, the blower may be arranged, for example, on a tractor configured to carry the device, wherein in this case, compressed air is provided to the device by the blower arranged on the tractor.

According to a preferred embodiment, a velocity of the air leaving the nozzle arrangement ranges from 5 km/h to 45 km/h, preferably from 8 km/h to 40 km/h. The velocity may, for example range from 5 km/h to 15 km/h, from 15 km/h to 30 km/h or from 30 km/h to 45 km/h. The mentioned speed range has been found to be beneficial to transport the pollen over a considerable distance while maintaining the viability of the pollen. It has been found to be beneficial that the air, when hitting pollen bearing wheat plants, comprises a velocity ranging from 10 km/h to 20 km/h. Thereby, also the natural wind which may be present at the agricultural field, may be taken into account. In other words, when the wind speed is rather high, the velocity of the additional air flow to be provided by the nozzle arrangement might be lower than in conditions at which the natural wind speed at the field is rather low.

According to another embodiment, the nozzle arrangement is a first nozzle arrangement and wherein the device comprises a second nozzle arrangement spaced apart from the first nozzle arrangement. Preferably, at least one nozzle arrangement is directed towards a certain pollen-producing crop row. By utilizing, for example, two nozzle arrangements, air may be blown towards two pollen-producing crop rows simultaneously when the device is moved over the agricultural field. Preferably, the device comprises 5 to 20 nozzle arrangements, in particular 10 nozzle arrangements. The device may also comprise 6, 7, 8, 9, 11, 12, 13, 14, 15, 16, 17, 18, 19 nozzle arrangements. By utilizing 10 nozzle arrangements, the device may blow air against, for example, 10 different rows of the pollen-producing crop plants.

In one embodiment, the carrier structure comprises at least one diagonal strut extending from a central front portion of the device diagonally outwards and backwards towards a back portion of the device, wherein at least two nozzle arrangements, in particular 3, 4, 5, 6 or more nozzle arrangements, are attached to the diagonal strut. Arranging the nozzle arrangements at a diagonal strut ensures that the nozzle arrangements can be spaced as required for a certain specific crop row spacing. Furthermore, the diagonal design ensures that the pollen travel distance and distribution is improved, since when moving the device, subsequent nozzle arrangements will participate in the uplifting and/or transport of the pollen. By arranging the nozzle arrangements on different positions on the diagonal strut, different row spaces can be accommodated.

In another embodiment of the invention, the diagonal strut is a first diagonal strut and wherein the carrier structure comprises a second diagonal strut extending from the center front portion of the device diagonally outwards and backwards towards the back portion of the device, wherein at least two nozzle arrangements, in particular 3, 4, 5, 6 or more nozzle arrangements, are attached to the diagonal strut, and wherein the diagonal struts extend in opposite outward directions. In other words, the two diagonal struts form a triangle or wedge with respect to each other. In this way, the overall pollen distribution is increased wherein the design allows to accommodate for different wind conditions and directions in the agricultural field. For example, it might be beneficial to only utilize nozzle arrangements arranged on either of the diagonal struts. It has been found to be beneficial for certain crops to avoid having a nozzle arrangement blow against the wind. In this regard, when the wind passes, for instance, from right to left, it may be beneficial to only utilize the nozzle arrangements on the left of the device blowing in a leftward direction with regard to the moving direction of the device.

In another embodiment, the carrier structure comprises a carrier strut arranged perpendicular to the longitudinal axis, wherein the first diagonal strut and/or the second diagonal strut are attached to the carrier strut, wherein the carrier strut and the diagonal strut form a triangular basic shape. Utilizing said carrier strut has been found to be beneficial to increase the overall rigidity of the device. According to another embodiment, additional pivotable struts are connected to the diagonal struts, wherein the pivotable struts can be brought into a transport position, in which the pivotable struts are aligned in parallel to or inwardly towards the longitudinal axis and an extended position at which the pivotable struts are aligned and extended outwardly in order to increase a total width of the device. In this way, it can be ensured that the device can be transported properly by reducing its overall width when the pivotable struts are in the transport position, wherein the overall width of the device can be increased on the field in order to accommodate for larger row spacings.

According to a preferred embodiment, the exhaust direction of the nozzle arrangements arranged at the first diagonal strut is tilted towards the first outward direction, in particular by an angle of 1° to 90°, preferably 45° to 90°, for example 5°, 10°, 15°, 20°, 25°, 30°, 35°, 40°, 45°, 50°, 55°, 60°, 65°, 70°, 75°, 80°, 85°, from the longitudinal axis, and/or wherein the exhaust direction of the nozzle arrangements arranged at the second diagonal strut is tilted towards the second outward direction, in particular by an angle of 1° to 90°, preferably 45° to 90°, for example 5°, 10°, 15°, 20°, 25°, 30°, 35°, 40°, 45°, 50°, 55°, 60°, 65°, 70°, 75°, 80°, 85°, from the longitudinal axis, in order to suspend pollen towards the direction of the female plants. In other words, considering a scenario having five nozzle arrangements attached to each diagonal strut, the nozzle arrangements attached to the right diagonal strut, seen in a moving direction of the device, will blow rightwards with regard to the longitudinal axis by the mentioned angles, wherein the nozzle arrangements attached to the left diagonal strut will blow leftwards with respect to the longitudinal axis by the mentioned angle range. In one embodiment, the device is aligned with its longitudinal axis between two central crop rows. Between those two central crop rows, two nozzle arrangements are arranged. One is preferably attached to the first diagonal strut blowing in a rightward direction towards the central right row and the other is attached to the left diagonal strut blowing towards the central left row. Between the other pairs of crop rows, only one nozzle arrangement is arranged. In this way, in total ten different crop rows can be affected by ten nozzle arrangements.

According to another embodiment, a valve, in particular a solenoid valve, is arranged between the nozzle arrangement and the blower, wherein each valve is configured to cut or allow an air flow from the blower to the nozzle arrangement, in particular to collectively cut or allow an air flow from the blower to the nozzle arrangements arranged at the first and/or second diagonal strut. In other words, according to one embodiment, each nozzle arrangement may be activated or deactivated individually. According to a preferred embodiment, however, only nozzle arrangements attached to either the first or second diagonal strut are grouped together so that either the nozzle arrangements attached to first and/or the second diagonal strut can be activated or deactivated collectively, in particular controlled from the tractor that carries the device.

In one embodiment, the nozzle arrangements are spaced apart from each other in the direction perpendicular to the longitudinal axis by a distance of 7 cm to 45 cm, preferably 14 cm to 30 cm, in order to accommodate for different row planting spacing. This is achieved, for example, by adjusting the position of the nozzle arrangements at the diagonal struts in order to place the nozzle arrangements in between two adjacent crop rows for different row planting spacings.

Preferably, the nozzle arrangements are releasable attached to the carrier structure such that the spacing and/or height of the nozzle arrangements can be adjusted. In this way, the device can be used flexibly for a number of different rows spacings and also plant types and heights. For example, the height of the nozzle arrangements can be adjusted to accommodate for certain crop species. In one embodiment, however, the height of the device with regard to the crops is adjusted by means the tractor carrying the device.

According to another embodiment, the nozzle arrangement, in particular each nozzle arrangement, comprises an air knife. Air knifes comprise an elongated nozzle that provides a uniform sheet of laminar air flow, also known as streamline flow. The air knife can be beneficially utilized to blow air in a defined direction and over an elongated nozzle area towards the crop plants. In particular, the air knife is a first air knife and wherein the nozzle arrangement, in particular each nozzle arrangement, optionally comprises a second air knife. The use of two air knives has been found to be beneficial to provide a uniform air flow to the crop plants and/or to increase the amount of pollen being released. In one embodiment, the vaporizing device is arranged in the middle of and above the two air knives. In another embodiment, the vaporizing device is contained in the air knife. In another embodiment, the vaporizing device may be integrated into an air providing system for providing air to the air knife, or may be located outside the air knife, e.g. at the air exhaust of the air knives. This has been found to be beneficial to increase the relative humidity of the air leaving the air knives as described above and reduce condensation. In one embodiment, a length of the air knives along a longitudinal axis thereof ranges from 10 cm to 40 cm, preferably 20 cm to 30 cm. The length may be 15 cm, 20 cm, 25 cm, 30 cm or 35 cm. By providing air knives with the above total length, plants, such as wheat plants, can be beneficially provided with the air knife air flow.

According to one embodiment, the air knife or the air knives are inclined with respect to a crop row vertical plain in particular by an angle of 1° to 45°, preferably 10°, 20°, 30°, 40°, in order to blow the air against the male (pollen-producing) crop row initially from the bottom and thereafter from the top of the air knife. Furthermore, the described alignment ensures that the pollen are blown upwards. Preferably, the angle is adjusted to the crop row spacing. For smaller crop row spacings, it may be beneficial to reduce the angle in order to avoid the air knives touching the crop rows. For larger crop rows spacings, the angle may be increased without touching the crop rows. In a preferred embodiment, the alignment and/or tilting of the air knives is conducted manually. In an alternative embodiment, the alignment and/or tilting of the air knives is conducted by means of at least one actuating device that is configured to align and/or tilt the air knives.

In one embodiment, the carrier structure comprises an interface configured for coupling the device to a tractor, in particular to a front receiving section of a tractor. When the device is attached to the tractor, the overall height of the device with respect to the crops can be adjusted by lifting the device upwards or downwards. In one embodiment, the tractor provides a water supply for the vaporizing device and/or air pressure for operating the nozzle devices or air knives. According to an alternative embodiment, an air compressor and/or a water tank including a water compressor is arranged on the device itself. Furthermore, energy for operating an air compressor or a water pump may be provided by a tractor power take-off (PTO).

In one embodiment, the device comprises a protection guard. The protection guard is preferably arranged adjacent to the air knife, in particular substantially in parallel to the air knife. Said guard is an optional component and may be utilized to protect the air knives from mechanical influences, particularly when the distance between plant rows is short. In one embodiment of the invention, the air knives have no protection guard.

In one embodiment, the device comprises a sensor unit, wherein the sensor unit comprises at least one of the following sensors: camera, satellite navigation system receiver, height sensor arrangement configured to determine a height of the crop rows relative to a vertical position of the pollen distribution device. The sensor unit may be part of a so-called “smart farming”—system. The sensor unit may be a central unit comprising the mentioned sensors or may be a distributed system comprising components at different positions on the device. A camera may be mounted on top of the device to provide a suitable overview and/or adjacent to (at least one of) the air knives or other parts of the device that require special attention from the device operator.

In one embodiment, the height sensor arrangement comprises two infrared sensors that are spaced vertically from each other. The infrared sensors can be utilized to adjust the height of the device with respect to the crop rows. When two infrared sensors that are arranged vertically above one another are utilized, the height of the device may be controlled such that the lower sensor is blocked by the crops passing along the sensors and the higher sensor receives an infrared signal which means that the sensor is not blocked by the plants. This setup corresponds to the preferred operating height h of the device. When both infrared sensors are blocked, the height of the device is too low. When both infrared sensors receive an infrared signal, the height of the device is too high. By controlling the height of the device such that the indication of the infrared sensors is as explained above, a robust height measurement and adjustment for the device is provided. With the help of the sensor signals, the device may be operated in an automated way wherein, for example, the height of the device is adjusted automatically. Also the correct positioning of the device with regard to male or female crop rows can be automated based on the camera signal and/or the satellite navigation system.

In one embodiment, the device further comprises a front bar arrangement, said front bar arrangement comprising a front bar which is arranged substantially perpendicular to the moving direction at a front portion of the device. The front bar is preferably foldable. In one embodiment, the front bar arrangement comprises at least one chain attached to the front bar, in particular wherein the chain is a first chain and wherein the front bar comprises at least one second chain spaced apart from the first chain. The chains are utilized to support hanging bars that stimulate the female plants when passing the device along the agricultural field. The chains have a length such that the hanging bars touch the female plant heads, e.g., the bars are positioned at the lower side of the air knives, or just below the heads/flowers. It needs to be noted that the use of the front bar arrangement is optional. It is preferred that the female plants are touched so as to stimulate flower opening, but are not damaged, broken or cut, by the hanging bars or chains. In one embodiment, the hanging bars only touch the rows of the bay of female plants adjacent to the male plants, and do not touch the male plants from which pollen is to be dispersed. In one embodiment, also the male plants can be touched by the hanging bars, e.g., when male flowers are not yet open (no anthers extruded), so as to stimulate anther extrusion.

In one embodiment, a wire support structure is arranged above the font bar. Preferably, the wire support structure comprises a central post extending from the front bar vertically upwards, wherein at least one wire is tensioned between the front bar and a top of the central post. In one embodiment, a wire extends from the top of the central post to either side of the bar. The support structure helps to avoid bending of the front bar.

In a preferred embodiment, the front bar comprises four or six chains. Two chains are preferably arranged at outer edges of the front bar and the other two or four chains are arranged in accordance with the bay width of the female plants in the field. In a preferred embodiment, the front bar arrangement comprises a chain mount configured to releasably mount the chain to the front bar and/or to adjust the position of the chain along the front bar. In this way, the positioning of the chains can be adjusted with regard to the field setup. In one embodiment, two chains are positioned at the outer edges, and two chains are positioned just outside the male plant row plot, so that the hanging bars only touch female plant rows.

In one embodiment, the device comprises an insulation cloth arranged between the tractor and plants in order to avoid the engine, or other parts that get hot in use and can transfer heat to the plants. The insulation cloth may comprise a roll that may be rolled-up (such as during transport) and may be fixed in the unrolled state to provide heat shielding. The support bar to which the air knives are releasably attached may comprise markings with pre-set row distances, for predetermined air knife positions for specific row spacings (between the male rows).

In another aspect, the invention relates to a pollen distribution device for crop plants in an agricultural field, the device comprising a carrier structure configured to be moved along rows of crop plants on the agricultural field in a moving direction, a blowing device attached to the carrier structure, wherein the blowing device comprises at least one nozzle arrangement configured to blow air against pollen bearing plants of the agricultural field.

Said aspect of the invention attains its object in that the pollen distribution device comprises a vaporizing device being separate from the blowing device for adding humidity, wherein the vaporizing device is configured to increase the relative humidity of the air surrounding the pollen distribution device (e.g., to between 60% and 80%) in order to ensure high relative humidity suitable for pollen viability preservation by atomizing the water particles.

The further aspect of the pollen distribution device takes advantage of the same benefits and preferred embodiments as the pollen distribution device according to the invention and vice versa. In this regard and in order to avoid unnecessary repetitions, reference is made to the above explanations.

Until now, the invention has been described with respect to the pollen distribution device. In another aspect, the invention relates to a use of a pollen distribution device according to any of the previous embodiments for distributing pollen while maintaining viability due to a reduction of dehydration of the following crop plants: wheat (Triticum spp.), in particular winter wheat and spring wheat, including Triticale, rice (Oryza sativa spp.), or rice genus, oat (Avena spp.), including Avena sativa, barley (Hordeum spp.), including Hordeum vulgare, corn (Zea spp.), including Zea mays, onions or leek (Allium spp.), including Allium cepa, carrots (Daucus spp.), including Daucus carota subsp. Sativus, as well as a plant from another genus or species where cross-pollination of pollen-receiving plants (such as male-sterile plants for crops with male and female reproductive organs) by pollen-producing plants is desired and/or a comparable inflorescence architecture suitable for mechanically assisted pollination.

The use of the pollen distribution device takes advantage of the same benefits and preferred embodiments as the pollen distribution device according to the invention and vice versa. In this regard and in order to avoid unnecessary repetitions, reference is made to the above explanations.

In a further aspect, the invention relates to a method for operating a pollen distribution device according to any of the previous embodiments. The method attains the object of the invention with the step: Moving the pollen distribution device along at least one row of crop plants of an agricultural field with a ground speed of 2.5 km/h to 15 km/h, preferably with a ground speed of 5 km/h to 10 km/h, in one embodiment twice a day, particularly short to one another, such as separated by 5 minutes to 30 minutes. By utilizing the above speed range, the cross-pollination potential of the device is maximized. The device may be moved along the rows of crop plants twice a day, wherein the first pass may stimulate gaping of the plants.

Preferably, the method further comprises a step of moving the pollen distribution device along the row of crop plants another time, wherein a time interval between the previous pass and the second pass is in particular one day, preferably two days. This has been found to be beneficial for improving cross-pollination. Furthermore, it can also be 2 passes on one day and 2 passes on another day, with the 2 passes on one day being short to one another, such as separated by 5 minutes to 30 minutes.

Preferably, the device is operated under the following conditions at the agricultural field: No rain, at least 30% of the ears of the male (pollen-producing) plants comprise pollen, in particular mature pollen (particularly, at least 30% of the ears of the male plants have anthers extruded), female plants receptive, in particular where in at least 30% of the ears of the female plants comprise gaping florets, no substantial cloud coverage, after morning dew has evaporated and before temperatures in the field rise above 20° C., preferably before reaching a temperature above 25° C. or 30° C. These conditions have been found to be beneficial for optimized cross-pollination-results.

Preferably, the size of the male plant plots fit the size of the tractor, in particular the distance between the wheels of the tractor, so that the wheels drive between male and female rows.

For a more complete understanding of the invention, the invention will now be described in detail with reference to the accompanying drawings. The detailed description will illustrate and describe what is considered as a preferred embodiment of the invention. It should of course be understood that various modifications and changes in form or detail could readily be made without departing from the spirit of the invention. It is therefore intended that the invention may not be limited to the exact form and detail shown and described herein, nor to anything less than the whole of the invention disclosed herein and as claimed hereinafter. Further, the features described in the description, the drawings and the claims disclosing the invention may be relevant for the invention considered alone or in combination. In particular, any reference signs in the claims shall not be construed as limiting the scope of the invention. The wording “comprising” does not exclude other elements or steps. The wording “a” or “an” does not exclude a plurality.

This invention will now be described with reference to the accompanying drawings, which illustrate, by way of example and not by way of limitation, one of several possible embodiments of the device as proposed herein, and wherein:

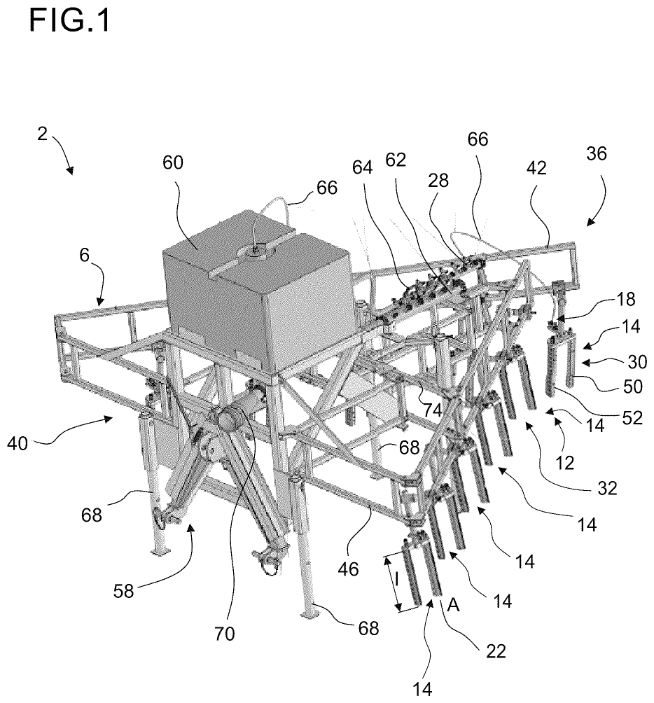

FIG. 1: shows a pollen distribution device according to an embodiment of the invention in a perspective view;

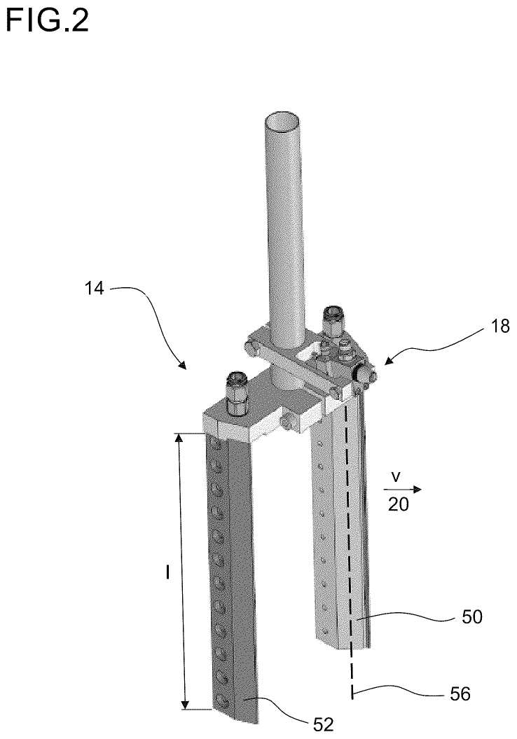

FIG. 2: shows a nozzle arrangement of the pollen distribution device according to the embodiment of FIG. 1 in a perspective view;

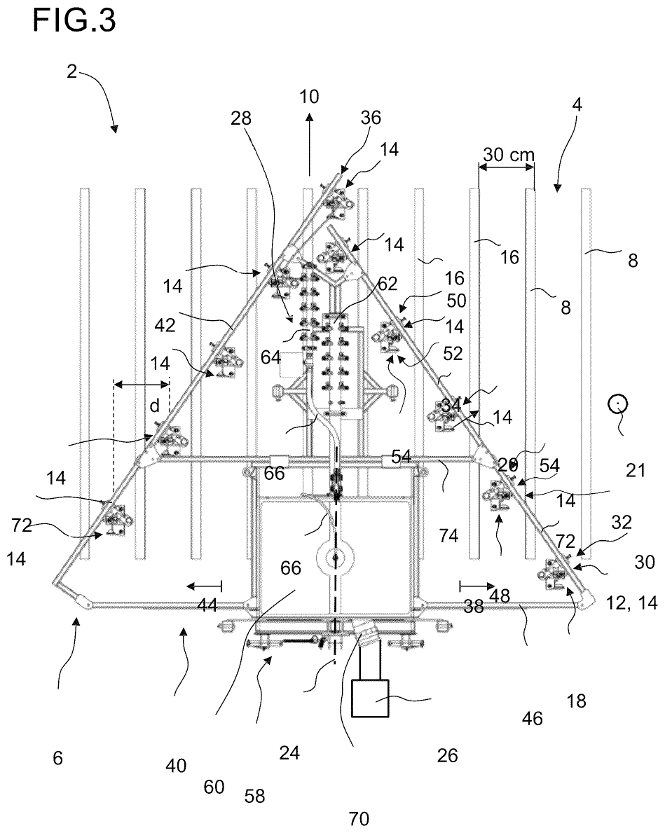

FIG. 3: shows the pollen distribution device according to the embodiment of FIG. 1 in a top view, wherein the device is adjusted to a larger crop row plant spacing;

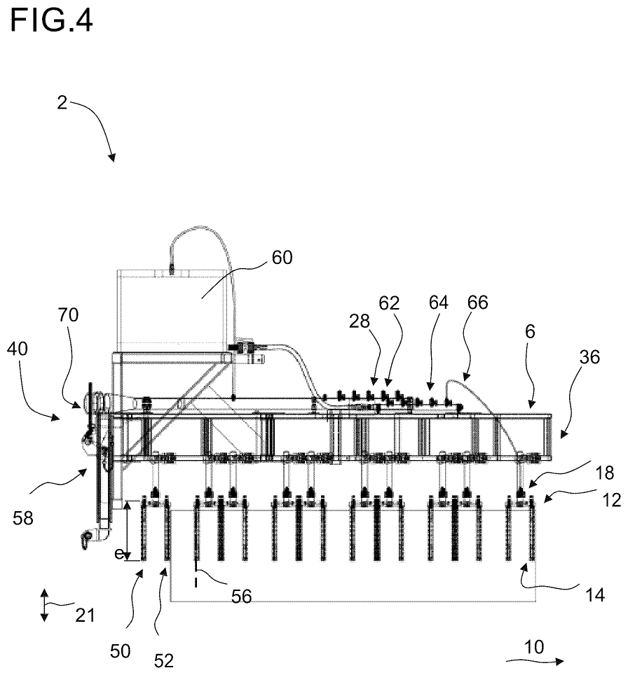

FIG. 4: shows the pollen distribution device according to the previous figures in a side view;

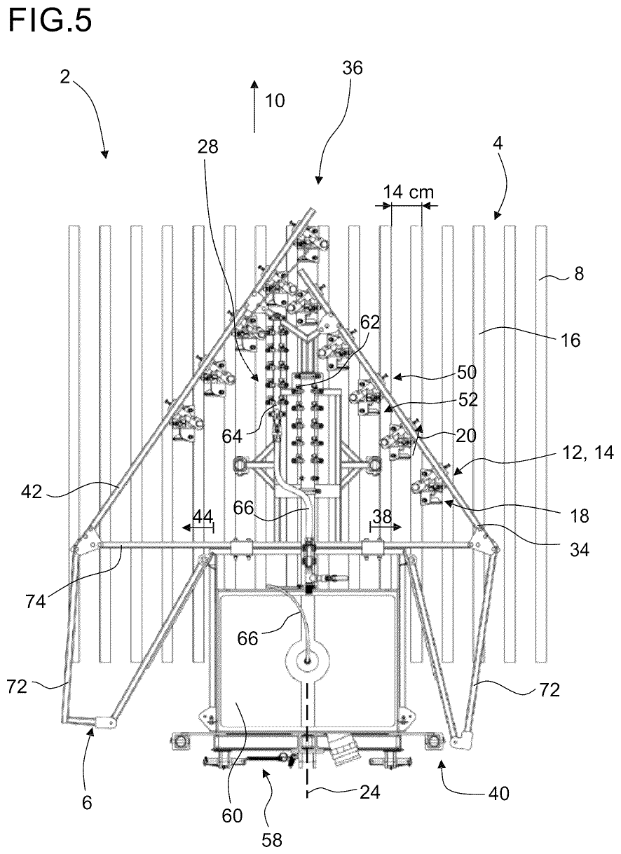

FIG. 5: shows the pollen distribution device according to the embodiment of FIG. 1 in a top view, wherein the device is adjusted to a smaller crop row plant spacing;

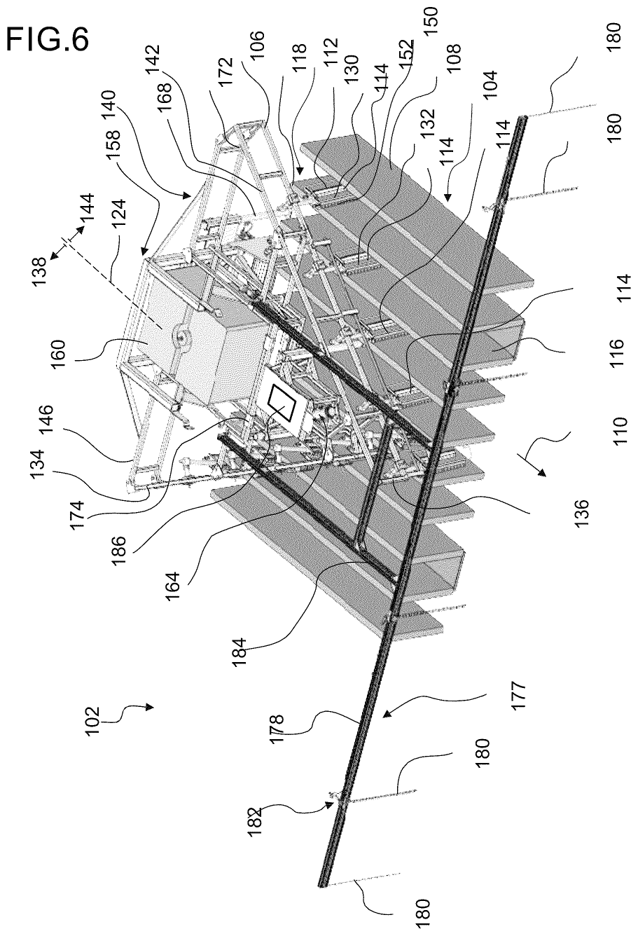

FIGS. 6-12: show an alternative embodiment of a pollen distribution device in various views and perspectives;

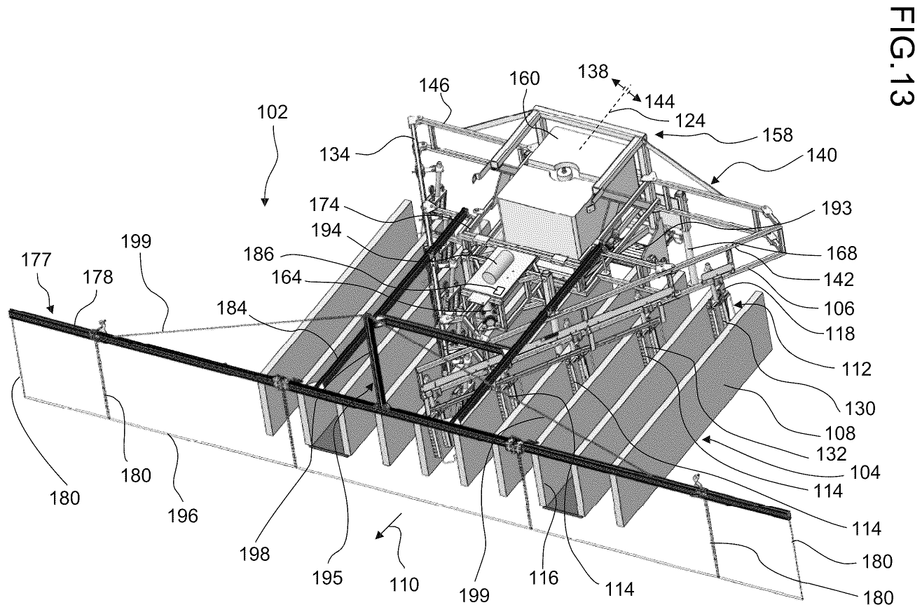

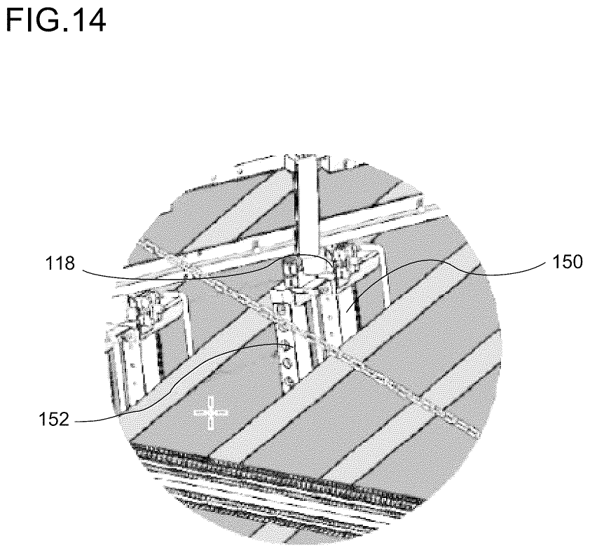

FIGS. 13, 14 show an alternative embodiment of a pollen distribution device in perspective views;

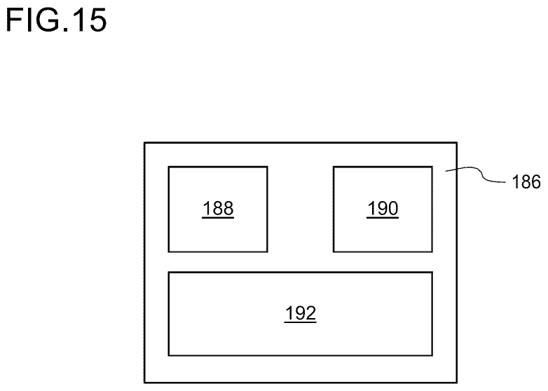

FIG. 15: shows an embodiment of sensor unit of the pollen distribution device according to FIGS. 6-14; and

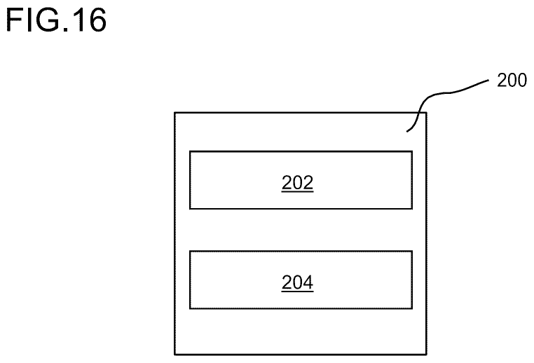

FIG. 16: shows an embodiment of a method according to the invention in a schematic view.

FIGS. 1, 3 and 4 show a pollen distribution device 2 for a crop row plant in an agricultural field 4, as shown in FIG. 3. The device 2 comprises a carrier structure 6. The carrier structure 6 is configured to be moved along rows 8 of crop plants of the agricultural field 4 in a moving direction 10, which is shown in FIG. 3. The pollen distribution device 2 furthermore comprises a blowing device 12. The blowing device 12 is attached to the carrier structure 6. The blowing device 12 comprises ten nozzle arrangements 14. The nozzle arrangements 14 are configured to blow air against pollen bearing plants 16 of the agricultural field 4 as shown in FIG. 3. The nozzle arrangements 14 comprise a vaporizing device 18. In one embodiment, this vaporizing device 18 is contained in each air nozzle of the nozzle arrangements 14 or in an air supply system that supplies air to the nozzle arrangements 14, so that the air leaving the nozzle arrangements has a higher humidity. The vaporizing device 18 is configured for adding humidity to the air leaving the nozzle arrangements 14. In particular, the vaporizing device 18 is configured to increase the relative humidity of the air leaving the nozzle arrangement 14 between 60% and 80%, in order to ensure high relative humidity suitable for pollen viability preservation by atomizing the water particles. In this regard, it is also made sure that the pollen is still viable without having the pollen becoming wet.

Exhaust directions 20 of the air leaving the nozzle arrangements 14 are inclined upwards with respect to a vertical direction 21 by an angle 22 of 1° to 45°, preferably 10°, in order to suspend or blow the pollen upwards. Furthermore, the exhaust direction 20 of the air leaving the nozzle arrangements 14 is tilted rightwards or leftwards with respect to a longitudinal axis 24 of the device 2, in particular outwards with respect to the longitudinal axis 24. The device 2 or a corresponding tractor comprises a blower 26, in particular a centrifugal blower 26. The blower 26 is fluidly connected to the nozzle arrangements 14. A velocity v, as shown in FIG. 2, of the air leaving the nozzle arrangement 30 ranges from 5 km/h to 45 km/h, preferably from 8 km/h to 40 km/h. The blower 26 is configured to adjust the velocity v of the air leaving the nozzle arrangements 14 so that air, when hitting the pollen bearing plants 16 comprises a velocity ranging from 10 km/h to 20 km/h in the case of wheat plants.

The nozzle arrangement 14 may be a first nozzle arrangement 30, wherein the device comprises a second nozzle arrangement 32 spaced apart from the first nozzle arrangement 30. In particular and as shown in the figures, the device 2 comprises ten nozzle arrangements 14 that are spaced from each other. The carrier structure 6 comprises a first diagonal strut 34. The first diagonal strut extends from a central front portion 36 of the device 2 diagonally outwards and backwards towards a back portion 40 of the device 2. Five nozzle arrangements 14 are attached to the first diagonal strut 34. The carrier structure 6 comprises a second diagonal strut 42 extending from the central front portion 36 of the device 2 diagonally outwards and backwards towards the back portion 40 of the device 2. Five nozzle arrangements 14 are attached to the second diagonal strut 42. The first diagonal strut 34 and the second diagonal strut 42 extend in opposite outward directions 38, 44, respectively, so as to form a wedge-shaped geometry.

The carrier structure 6 comprises a carrier strut 74. The carrier strut 74 is arranged perpendicular to the longitudinal axis 24. The first diagonal strut 34 and the second diagonal strut 42 are attached to the carrier strut 46. The carrier strut 46 and the diagonal struts 34, 42 from a triangular basic shape. Furthermore, additional pivotable struts 72 are connected to the diagonal strut 34, 42. The pivotable struts 72 can be brought into a transport position at which the pivotable struts 72 are aligned in parallel to or inwardly towards the longitudinal axis 24. The pivotable struts 72 can furthermore be brought into an extended position at which the pivotable struts 72 are aligned outwardly in order to increase a total width of the device 2 as shown in FIG. 3. The transport position, which can also be utilized for smaller row spacings, is shown in FIG. 5. The exhaust direction 20 of the nozzle arrangements 14 arranged at the first diagonal strut 34 is tilted towards the first outward direction 38, which means to the right in the plane of the drawing. The tilting angle 48 is 1° to 90°, preferably 45° to 90°, from the longitudinal axis 24. The exhaust direction 20 of the nozzle arrangements 14 arranged at the second diagonal strut 42 is tilted towards a second outward direction 44, in particular by an angle 48 of 1° to 90°, preferably 45° to 90°, from the longitudinal axis 24. In this way, the pollen are suspended or blown towards the direction of the female or pollen-receiving plants.

Valves 28, in particular solenoid valves 28, are arranged between the nozzle arrangement 14 and blower 26. Each valve 28 is configured to cut or allow an air flow from the blower 26 to the nozzle arrangement 14, in particular to collectively cut or allow an air flow from the blower 26 to the nozzle arrangements 14 arranged at the first and/or the second diagonal strut 34, 42. In particular, an air flow from the blower 26 to all nozzle arrangements 14 arranged on either the first or the second diagonal strut 34, 42 can be collectively activated or deactivated. In this way, the device 2 can accommodate to different wind conditions present at the agricultural field 4. The nozzle arrangements 14 are spaced apart from each other in the direction perpendicular to the longitudinal axis 24 by a distance d of 10 cm to 45 cm, preferably 14 cm to 30 cm. For example, in the embodiment shown in FIG. 3, the nozzle arrangements 14 are spaced by approximately 30 cm. In the embodiment shown in FIG. 5, the nozzle arrangement are spaced corresponding to a spacing of the rows 8 by about 14 cm. The nozzle arrangements 14 are releasable attached to the carrier structure 6 such that the spacing and/or the height of the nozzle arrangements 14 with regard to the carrier structure 6 can be adjusted.

Each nozzle arrangement 14 comprises a first air knife 50 and a second air knife 52. The use of the second air knife 52 is optional. The air knives 50 are inclined with respect to a crop row 8 vertical plane by an angle 54 of in particular 10°. The angle 54 may range between 1° and 45°. In this way, the air is blown against the crop row 8 initially from the bottom of the air knife 50 and thereafter from the top of the air knife 50. Hence, the pollen are blown upwards. The air knives 50 furthermore comprise a length l. The length l along a longitudinal axis 56 of the air knives 50 ranges from 10 cm to 40 cm wherein a preferred range is between 20 cm and 30 cm. Furthermore, the carrier structure 60 comprises an interface 58. The interface 58 is configured for coupling the device 2 to a tractor (not shown), in particular to a front receiving section of a tractor.

Furthermore, a water tank 60 is attached to the carrier structure 6. The water tank 60 comprises water utilized for the vaporizing device 18 and may comprise a pump. With the help of hoses 66, the water tank is fluidly connected to water manifold 64 and, with the help of another hose 66, to the nozzle arrangements 14 and towards the vaporizing device 18. Pressurized air for operating the air knives 50 is provided in the embodiment of FIG. 1 by means of a hose 70 from an external source, such as a tractor comprising a compressor (not shown). It is guided from the hose 70 towards an air manifold 62 and towards the air knives 50. In an alternative embodiment, as shown in FIG. 3, a centrifugal blower 26 may be attached to the device 2 providing compressed air to the air manifold 62. The device 2 furthermore comprises struts 72 that can be pivoted in order to adjust the overall width of the device 2. Moreover, the device comprises a number of posts 68 that can be utilized to arrange the device 2 on the ground, for example when the device 2 is not utilized or in order to couple the device 2 with the help of the coupling interface 58 to a tractor (not shown).

As already briefly described, FIG. 2 shows a more detailed perspective of a nozzle arrangement 14. The nozzle arrangement 14 comprises a first air knife 50 and a second air knife 52. The exhaust direction 20 of air leaving the air knife 50 is shown in the figure. The air leaves the air knife 50 with a velocity v. Above and in between of the air knives 50, 52, the vaporizing device 18 is arranged. In FIG. 4, a side view of the embodiment of the device according to FIG. 3 is shown.

FIGS. 6 to 9 show an alternative embodiment of a pollen distribution device 102. In FIG. 6, the pollen distribution device 102 is shown in a perspective view. The device 102 comprises a carrier structure 106. The carrier structure 106 is configured to be moved along rows 108 of crop plants of an agricultural field 104 in a moving direction 110. As described with regard to the first embodiment in the previous figures, the pollen distribution device 102 furthermore comprises a blowing device 112. The blowing device 112 is attached to the carrier structure 106.

The blowing device 112 comprises ten nozzle arrangements 114. The nozzle arrangements 114 are configured to blow air against pollen bearing plants 116 of the agricultural field 104. The nozzle arrangements 114 comprise a vaporizing device 118.

In one embodiment, this vaporizing device 118 is contained in each air nozzle of the nozzle arrangements 114 or in an air supply system that supplies air to the nozzle arrangements 114, so that the air leaving the nozzle arrangement has a higher humidity. Alternatively, the nozzle arrangements 114 may generate atomized water particles. In an alternative embodiment, the vaporizing device may be separated from the blowing device.

In general, the vaporizing device 118 is configured for adding humidity to the air leaving the nozzle arrangements 114 or may provide atomized liquid without having to add additional air. In particular, in one embodiment the vaporizing device 118 is configured to increase the relative humidity of the air leaving the nozzle arrangement 114 or the air hitting the pollen bearing plants (such as to a humidity between 60% and 80%), in order to ensure high relative humidity suitable for pollen viability preservation by atomizing water particles. In this regard, in one embodiment, it is also made sure for the device 102 that the pollen is still viable without the pollen becoming wet.

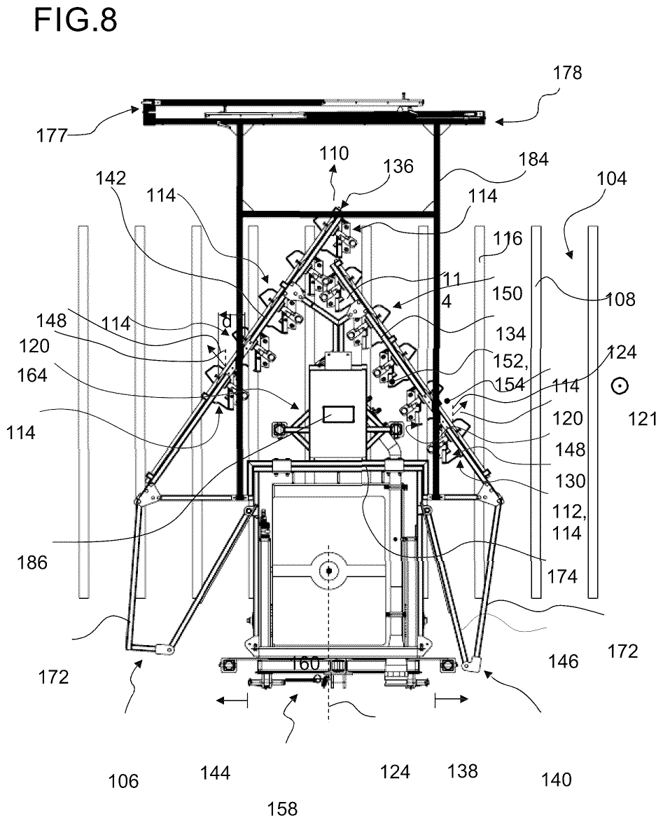

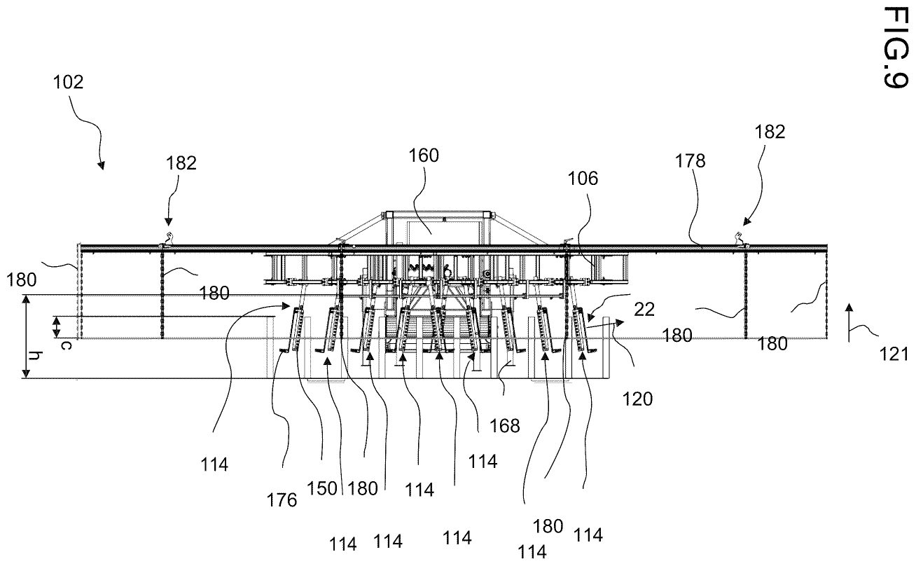

As shown in FIGS. 8 and 9, exhaust directions 120 of the air leaving the nozzle arrangements 114 are inclined upwards with respect to a vertical direction 121 by an angle 22 of 1° to 45°, preferably 10°, in order to blow the pollen upwards. Furthermore, the exhaust direction 120 of the air leaving the nozzle arrangements 114 is tilted rightwards or leftwards with respect to a longitudinal axis 124 of the device 102, in particular outwards with respect to the longitudinal axis 124. The device 102 or a corresponding tractor comprises a blower 126 not shown on FIG. 8 or 9, in particular a centrifugal blower 126. The blower 126 is fluidly connected to the nozzle arrangements 114. A velocity v of the air leaving the nozzle arrangement 114 ranges from 5 km/h to 45 km/h, preferably from 8 km/h to 40 km/h. The blower 126 is configured to adjust the velocity v of the air leaving the nozzle arrangements 114 so that air, when hitting the pollen bearing plants 116 comprises a velocity ranging from 10 km/h to 20 km/h in the case of wheat plants.

The nozzle arrangement 114 may be a first nozzle arrangement 130, wherein the device 102 comprises a second nozzle arrangement 132 spaced apart from the first nozzle arrangement 130. In particular and as shown in the figures, in one embodiment the device 102 comprises ten nozzle arrangements 114 that are spaced from each other. The carrier structure 106 comprises a first diagonal strut 134. The first diagonal strut extends from a central front portion 136 of the device 102 diagonally outwards and backwards towards a back portion 140 of the device 102. Five nozzle arrangements 114 are attached to the first diagonal strut 134. The carrier structure 106 comprises a second diagonal strut 142?extending from the central front portion 136 of the device 102 diagonally outwards and backwards towards the back portion 140 of the device 102. Five nozzle arrangements 114 are attached to the second diagonal strut 142. The first diagonal strut 134 and the second diagonal strut 142 extend in opposite outward directions 138, 144, respectively, so as to form a wedge-shaped geometry.

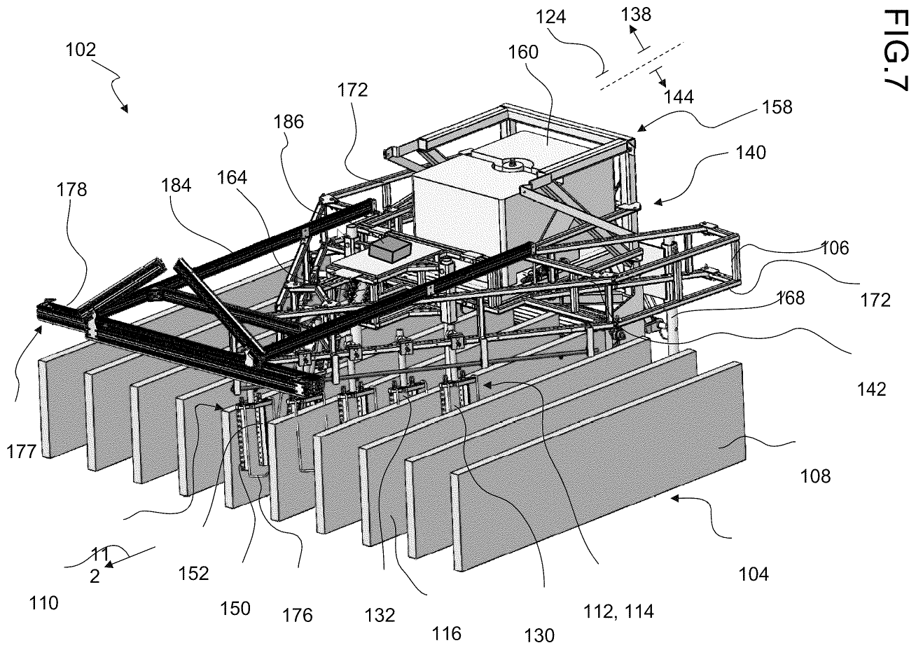

The carrier structure 106 comprises a carrier strut 174. The carrier strut 174 is arranged perpendicular to the longitudinal axis 124. The first diagonal strut 134 and the second diagonal strut 142 are attached to the carrier strut 174. The carrier strut 146 and the diagonal struts 134, 142 from a triangular basic shape. Furthermore, additional pivotable struts 172 are connected to the diagonal strut 134, 142. The pivotable struts 172 can be brought into a transport position as shown in FIG. 7 at which the pivotable struts 172 are aligned in parallel to or inwardly towards the longitudinal axis 124. The pivotable struts 172 can furthermore be brought into an extended position at which the pivotable struts 172 are aligned outwardly in order to increase a total width of the device 102 as shown in FIG. 6. The transport position, which can also be utilized for smaller row spacings, is shown in FIG. 7. The exhaust direction 120 of the nozzle arrangements 114 arranged at the first diagonal strut 134 is tilted towards the first outward direction 138. The tilting angle 148 is 1° to 90°, preferably 45° to 90°, from the longitudinal axis 124. The exhaust direction 120 of the nozzle arrangements 114 arranged at the second diagonal strut 142 is tilted towards a second outward direction 144, in particular by an angle 148 of 1° to 90°, preferably 45° to 90°, from the longitudinal axis 124. In this way, the pollen are blown towards the direction of the female or pollen-receiving plants.

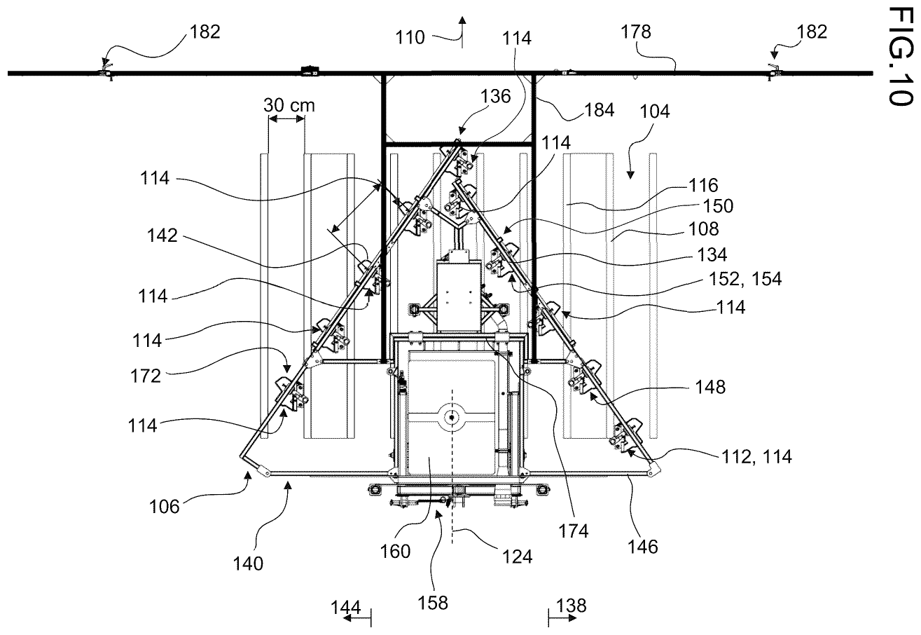

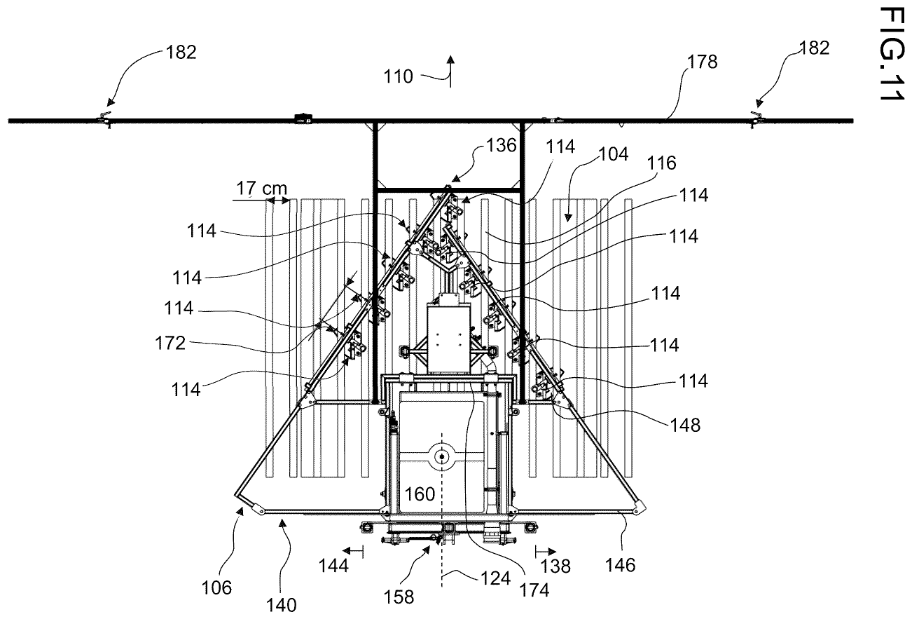

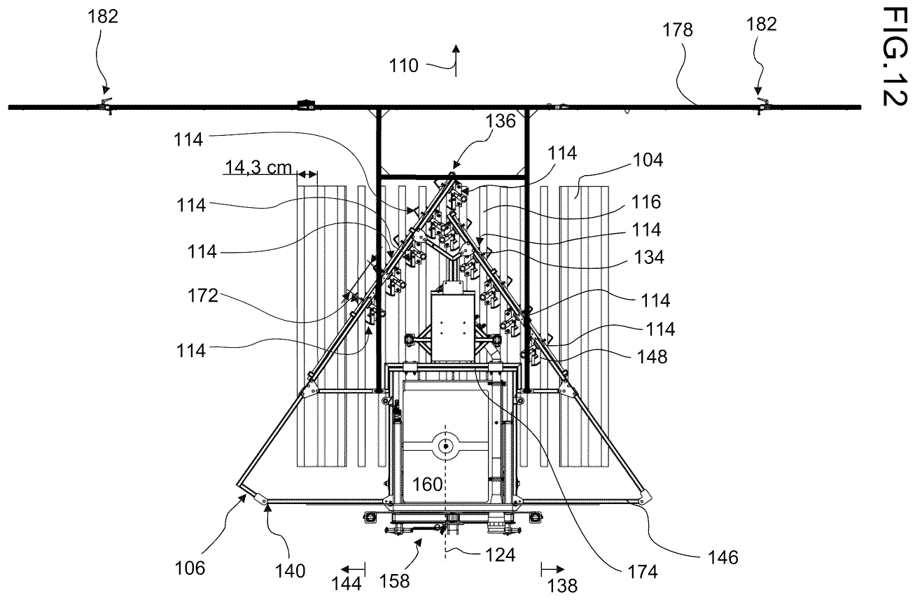

The nozzle arrangements 114 are spaced apart from each other in the direction perpendicular to the longitudinal axis 124 by a distance d (see FIG. 8) of 10 cm to 45 cm, preferably 14 cm to 30 cm. For example, in the configuration shown in FIG. 10, the nozzle arrangements 114 are spaced by approximately 50 cm to accommodate for a row spacing of 30 cm. In the configuration shown in FIG. 11, the nozzle arrangements 114 are spaced by approximately 30 cm to accommodate for a row spacing of 17 cm. In the configuration shown in FIG. 12, the nozzle arrangements 114 are spaced by approximately 25 cm to accommodate for a row spacing of 14.3 cm. The nozzle arrangements 114 are releasable attached to the carrier structure 106 such that the spacing and/or the height of the nozzle arrangements 114 with regard to the carrier structure 106 can be adjusted.

In one embodiment, each nozzle arrangement 114 comprises a first air knife 150 and a second air knife 152. The use of the second air knife 152 is optional. The air knives 150 are inclined with respect to a crop row 108 vertical plane upwards. The angle may range between 1° and 45°. In this way, the air is blown against the crop row 108 initially from the bottom of the air knife 150 and thereafter from the top of the air knife 150. Hence, the pollen are blown upwards. Furthermore, the carrier structure 106 comprises an interface 158. The interface 158 is configured for coupling the device 102 to a tractor (not shown), in particular to a front receiving section of a tractor. The air knife 150 is inclined with respect to a crop row 108 vertical plane by an angle 154.

Furthermore, a water tank 160 is attached to the carrier structure 106. The water tank 160 comprises water utilized for the vaporizing device 118 and may comprise a pump 194 as shown in FIG. 13. The water tank is ideally flat, so as to improve visibility of the driver over the field and the pollen distribution device. The water tank is fluidly connected to the water manifold 164 that is fluidly connected with hoses to the nozzle arrangements 114, in particular to the vaporizing device 118. Pressurized air for operating the air knives 150 is provided by means of an external source, such as a tractor comprising a compressor (not shown). Moreover, the device 102 comprises a number of posts 168 that can be utilized to arrange the device 102 on the ground, for example when the device 102 is not utilized or in order to couple the device 102 with the help of the coupling interface 158 to a tractor (not shown). FIG. 14 shows the vaporizing device 118 arranged above the air knifes 150, 152.

Optionally, the device 102 furthermore comprises a protection guard 176 arranged adjacent to at least, some or all of the air knives 150, 152. The protection guard 176 is arranged substantially in parallel to the air knives 150, 152. It needs to be noted that the protection guards 176 fulfil a protection function for the air knives 150, 152, only. They do not have a pollen release functionality.

In addition, the device 102 comprises a sensor unit 186 that is illustrated as a block diagram in FIG. 15. The sensor unit 186 comprises at least one of the following sensors: Camera 188, satellite navigation systems receiver 190, height sensor arrangement 192 configured to determine a height of the crop rows 108 relative to a vertical position of the pollen distribution device 102. In the figures, the sensor unit 186 is schematically shown adjacent to the water tank 160. However, the sensor unit 186 itself as well as the sensors 188 to 192 may be arranged on any convenient position on the device 102.

The camera 188 may be mounted on top of the device 102 and/or adjacent to at least one of the air knives 150, 152 or other parts of the device 102 that should be monitored by, for example, an operator of the device 102. The sensor unit 186 may also comprise more than one camera 188. The height sensor arrangement 192 comprises two infrared sensors that are spaced vertically from each other. The height sensor arrangement 192 is utilized to allow for an height adjustment of the device 102 with regard to a height of the crop rows 108. In one embodiment, the height of the device 102 is controlled such that the lower of the two infrared sensors is always blocked by the respective crops of the crop row 108, wherein the higher sensor receives an infrared signal. This means that, for example, when both sensors are blocked by the crops, the device 102 is moved upwards until the higher sensor receives an infrared signal and the lower sensor is blocked by the crops. In case both sensors receive an infrared signal, the device 102 is arranged too high with regard to the crop rows and is lowered until the lower infrared sensor does not receive an infrared signal anymore.

The device 102 furthermore optionally comprises a front bar arrangement 177. Said front bar arrangement 177 comprises a front bar 178. The front bar 178 is arranged substantially perpendicular to the moving direction 110 at a front portion 136 of the device 102, and is fixed to front bar support structure 184. The front bar 178 is foldable and unfoldable. In FIG. 6, the front bar 178 is shown in an unfolded position which is the operating position. In FIG. 7, the front bar 178 is shown in a folded position. The front bar arrangement 177 comprises chains 180 attached to the front bar 178. The spacing of the chains 180 is variable and can be adjusted with regard to the spacing of the crop rows 116. In order to achieve this, the front bar arrangement 177 comprises a chain mount 182. The chain mount 182 is configured to releasably mount the chain 180 to the front bar 178 and/or to adjust the position of the chain 180 along the front bar 178. The chains 180 are utilized to carry horizontal bars 196 that stimulate female plant heads/flowers, preferably only female (male sterile) and not the pollen-providing male plants. Therefore, the device 102 may pass the plants more than once, such as twice, or three or four times. In a first pass (when the air knives may or may not be operational), the horizontal bars hanging from the chains 180 are utilized to stimulate the females. Some time after said first pass (such as in 15 or 30 minutes, but ideally during the same application morning), a second pass can be done with the air knives operational, and the front bar in folded or in unfolded position. As shown in FIG. 9, the chains 180 comprise a chain overlap c which ensures that the chains 180 touch the female plant heads/flowers, approximately at the lower height of the air knives 114, or just below the female heads/flowers.

FIG. 13 shows an embodiment in which a wire support structure 195 is arranged above the font bar 178. The wire support structure 195 comprises a central post 198 extending from the front bar 178 vertically upwards. The wire support structure 195 also comprises two wires 199 that are tensioned between the front bar 178 and a top of the central post 198 to either side of the front bar 178. The support structure 195 helps to avoid bending of the front bar 178. The pollen distribution device 102 further comprises a water pump 194 that is arranged adjacent the water tank 160. Further, the pollen distribution device 102 comprises a heat protector or insulation cloth 193. The heat protector or insulation cloth 193 is arranged between the tractor (not shown) and the plants. The heat protector or insulation cloth 193 may be contained in a roller comprising the rolled-up insulation cloth (that can be unrolled and attached at the sides and behind the engine (so as to cover the engine), in order to provide heat shielding. The insulation cloth is of a smooth heat-shielding material that does not get electrically charged by static electricity, and is of a material that does not attract or retain pollen.

FIG. 16 shows an embodiment of a method 200 for operating the pollen distribution device 2 which can also be used with any pollen distribution device according to the invention, such as pollen distribution device 102 herein. The method 200 comprises the step of moving 202 the pollen distribution device 2, 102 along at least one row 8, 108 of crop plants of an agricultural field 4, 104 with a ground speed of 2.5 km/h to 15 km/h, preferably with a ground speed of 5 km/h to 10 km/h, in particular twice a day, such as twice a day on one day. A first move of the device 2, 102 along the row 8, 108 may be utilized to stimulate gaping of the crop plants. Also, the female plants may be stimulated by the horizontal bars hanging from the chains of the front bar 178. The method 200 furthermore comprises a step of moving 202 the pollen distribution device 2, 102 along the row 8, 108 of crop plants another time, wherein a time interval between previous pass and the present pass is in particular one day, preferably two days. The device 2, 102 is operated in particular under the following conditions at the agricultural field: No rain, at least 30% of the ears of the male plants comprise pollen, female plants receptive, in particular wherein at least 30% of the ears of the female plants comprise gaping florets, no substantial cloud coverage, after morning dew has evaporated and before temperatures in the field rise above 20° C., preferably before reaching a temperature above 25° C. or 30° C. A second pass may be conducted at each different day, close after the first pass to stimulate gaping, so there are two passes on the first day and two passes on the later day, particularly short to one another, such as separated by 5 minutes to 30 minutes.

Subsequently, initial indoor test results that have been conducted to provide a proof-of-concept will be described. Under indoor conditions, a system comprising an air knife and a spraying device as proposed in the claimed invention has been passed along wheat plants that were in a pollen release state. Petri dishes with PDA growing medium (Potato Dextrose Agar) were arranged at different distances from the wheat plants and the amount and viability of the pollen received by the petri dishes was analysed.

Exemplary results are shown in Table 1 for a test scenario. The petri dishes were spaced in a blowing direction of the air knife that was at an angle so that pollen was blown towards the petri dishes and lifted upward. Each position was spaced from the subsequent position by 500 mm in a longitudinal direction. At some positions, two petri dishes were provided on the right of the surface and the left of the surface. All other positions comprise only one petri dish centrally arranged on the surface.

Although the indoor conditions were suboptimal (artificial light and heated (dry) indoor space) and the amount of pollen generated at this first trial was low, pollen were distributed between 0.5 m and 4 m from the plants while the viability was—for most of the petri dishes that received pollen—50% and higher. Pollen viability was determined using standard methods by counting the number of pollen dead and viable in a fixed area observed with Microscope USB Dino-Lite. The exemplary results are summarized in the following Table 1:

| Distance of | Amount of pollen | |
|---|---|---|
| petri dish from | per petri dish | Viability of |
| plants [cm] | (surface: 63 cm²) | pollen [%] |
| 0 | 70 | 50 |
| 50 | 0 | 0 |
| 50 | ~5 | 50 |
| 100 | ~5 | 66 |
| 150 | ~10 | 20 |
| 150 | ~5 | 66 |
| 200 | ~5 | 66 |
| 250 | 0 | 0 |
| 250 | ~5 | 50 |
| 300 | ~5 | 50 |
| 350 | 0 | 0 |
| 400 | ~3 | 20 |
| 450 | 0 | 2 |

Based on the above data, a first cautious conclusion may be drawn such that pollen can be distributed over a considerable range of about 4 meters with the use of air knifes under these settings, while increasing the air humidity by using a spraying device helps to ensure that a considerable amount of the pollen remains viable.

### LIST OF REFERENCES

2 Pollen distribution device
4 agricultural field
6 carrier structure
8 rows of crop plants
10 moving direction
12 blowing device
14 nozzle arrangement
16 pollen bearing plants
18 vaporizing device
20 exhaust direction
21 vertical direction
22 vertical inclination angle
24 longitudinal axis
26 centrifugal blower
28 solenoid valve
30 first nozzle arrangement
32 second nozzle arrangement
34 (first) diagonal strut
36 central front portion
38 first outward direction
40 back portion of the device
42 second diagonal strut
44 second outward direction
46 strut
48 outward tilting angle
50 (first) air knife
52 second air knife
54 air knife inclination angle
56 air knife longitudinal axis
58 coupling interface
60 water tank including compressor
62 air manifold
64 water manifold
66 hose
68 posts
70 hose
72 additional pivotable struts
74 carrier strut
102 pollen distribution device
104 agricultural field
106 carrier structure
108 rows of crop plants
110 moving direction
112 blowing device
114 nozzle arrangement
116 pollen bearing plants
118 vaporizing device
120 exhaust direction
121 vertical direction
124 longitudinal axis
130 first nozzle arrangement
132 second nozzle arrangement
134 (first) diagonal strut
136 central front portion
138 first outward direction
140 back portion of the device
142 second diagonal strut
144 second outward direction
146 strut
148 outward tilting angle
150 (first) air knife
152 second air knife
154 air knife inclination angle
158 coupling interface
160 water tank
164 water manifold
168 posts
172 additional pivotable struts
174 carrier strut
176 protection guard
177 front bar arrangement
178 foldable front bar
180 chain
182 chain mount
184 front bar support
186 sensor unit
188 camera
190 satellite navigation system receiver
192 height sensor arrangement
193 heat protector/insulation cloth
194 water pump
195 wire support structure
196 horizontal bar
198 post of the wire support structure
199 wire
200 operating method
202 moving the device along at least one row of crop plants
204 moving the device along at least one row of crop plants a second time or day
v exhaust air velocity
d nozzle arrangement spacing
l length of air knife
h operating height
c chain overlap

1. A pollen distribution device comprising:
a carrier structure configured to be moved along rows of crop plants of an agricultural field in a moving direction, and
a blowing device attached to the carrier structure, wherein the blowing device comprises at least one nozzle arrangement configured to blow air against pollen bearing plants of the agricultural field,
wherein the nozzle arrangement comprises a vaporizing device for adding humidity to the air leaving the nozzle arrangement, wherein the vaporizing device is configured to increase the relative humidity of the air leaving the nozzle arrangement between 60% and 80% to ensure high relative humidity suitable for pollen viability preservation by atomizing the water particles.

2. The device according to claim 1,
wherein an exhaust direction of the air leaving the nozzle arrangement is inclined upwards with respect to a vertical direction by an angle of 10 to 45°, in order to suspend the pollen upwards and/or
wherein an exhaust direction of the air leaving the nozzle arrangement is tilted rightwards or leftwards with respect to a longitudinal axis of the device, or outwards with respect to the longitudinal axis.

3. The device according to claim 1,
wherein the device comprises a blower, in particular a centrifugal blower, the blower being fluidly connected to the nozzle arrangement, and
wherein a velocity (v) of the air leaving the nozzle arrangement ranges from 5 km/h to 45 km/h, from 8 km/h to 40 km/h or from 10 to 20 km/h.

4. The device according to claim 1, wherein the nozzle arrangement is a first nozzle arrangement and wherein the device comprises a second nozzle arrangement spaced apart from the first nozzle arrangement, or wherein the device comprises 5 to 20 nozzle arrangements.

5. The device according to claim 1, wherein the carrier structure comprises at least one diagonal strut extending from a central front portion of the device diagonally outwards and backwards towards a back portion of the device, and wherein at least two nozzle arrangements, are attached to the diagonal strut.

6. The device according to claim 5,
wherein the diagonal strut is a first diagonal strut and wherein the carrier structure comprises a second diagonal strut extending from the central front portion of the device diagonally outwards and backwards towards the back portion of the device, wherein at least two nozzles arrangements, are attached to the second diagonal strut, and wherein the diagonal struts extend in opposite outward directions.

7. The device according to claim 5,
wherein the carrier structure comprises a carrier strut arranged perpendicular to the longitudinal axis, wherein the first diagonal strut (34, 134) and/or the second diagonal strut are attached to the carrier strut wherein the carrier strut and the diagonal struts form a triangular basic shape, and
wherein additional pivotable struts are connected to the diagonal struts, wherein the additional pivotable struts can be brought into a transport position, at which the pivotable struts are aligned in parallel to or inwardly towards the longitudinal axis and an extended position at which the pivotable struts are aligned outwardly in order to increase a total width of the device.

8. The device according to claim 5,
wherein the exhaust direction of the nozzle arrangements arranged at the first diagonal strut is tilted towards the first outward direction by an angle of 1° to 90°, from the longitudinal axis,
and/or wherein the exhaust direction of the nozzle arrangements arranged at the second diagonal strut is tilted towards the second outward direction by an angle of 1° to 90°, from the longitudinal axis, in order to suspend the pollen towards the direction of female plants.

9. The device according to claim 1, wherein a valve is arranged between the nozzle arrangement and the blower, wherein each valve is configured to cut or allow an airflow from the blower to the nozzle arrangement, or to collectively cut or allow an airflow from the blower to the nozzle arrangements arranged at the first and/or the second diagonal strut.

10. The device according to claim 4,
wherein the nozzle arrangements are spaced apart from each other in the direction perpendicular to the longitudinal axis by a distance (d) of 7 cm to 45 cm, in order to accommodate for different row planting spacing and/or
wherein the nozzle arrangements are releasable attached to the carrier structure such that the spacing (d) and height of the nozzle arrangements can be adjusted.

11. The device according to claim 1,
wherein the nozzle arrangement comprises an air knife wherein the air knife is a first air knife and wherein the nozzle arrangement optionally comprises a second air knife.

12. The device according to claim 11,
wherein the air knife is inclined with respect to a crop row vertical plane by an angle of 1° to 45°, in order to blow the air against the crop row initially from the bottom and thereafter from the top of the air knife and/or
wherein a length of the air knife along a longitudinal axis thereof ranges from 10 cm to 40 cm.

13. The device according to claim 1, wherein the carrier structure comprises an interface configured for coupling the device to a tractor, or to a front receiving section of tractor.

14. The device according to claim 11, further comprising a protection guard arranged adjacent to the air knife, wherein the protection guard is arranged substantially in parallel to the air knife.

15. The device according to claim 1, further comprising a sensor unit, wherein the sensor unit comprises at least one of the following sensors:
camera,
satellite navigation system receiver, and or
height sensor arrangement configured to determine a height of the crop rows relative to a vertical position of the pollen distribution device.

16. The device according claim 15, wherein the camera is mounted on top of the device and/or adjacent to at least one of the air knifes.

17. The device according to claim 1, further comprising a front bar arrangement, said front bar arrangement comprising a front bar which is arranged substantially perpendicular to the moving direction at a front portion of the device, a wherein the front bar is foldable.

18. The device according claim 17, wherein the front bar arrangement comprises at least one chain attached to the front bar, wherein the chain is a first chain and wherein the front bar arrangement comprises at least one second chain spaced from the first chain and/or
wherein the front bar arrangement comprises a chain mount configured to releasably mount the chain to the front bar and/or to adjust the position of the chain along the front bar.

19. A method for distributing pollen comprising using the pollen distribution device according to claim 1 while maintaining pollen viability due to a reduction of dehydration of the following crop plants:
wheat (Triticum spp.),
rice (Oryza sativa spp.), or rice genus,
oat (Avena spp.),
barley (Horedeum spp.),
corn (Zea spp.),
onions or leek (Allium spp.),
carrots (Daucus spp.),
and/or a plant from another genus or species where cross-pollination of pollen-receiving plants by pollen-producing plants is desired and/or having a inflorescence architecture suitable for mechanically assisted (cross-)pollination.

20. A method for operating a pollen distribution device according to claim 1, the method comprising:
moving the pollen distribution device along at least one row of crop plants of an agricultural field with a ground speed of 2.5 km/h to 15 km/h.

21. The method of claim 20, further comprising:
moving the pollen distribution device along the row of crop plants at another time, wherein a time interval between the previous pass and the present pass is one day, and/or wherein the device is operated under the following conditions at the agricultural field:
no rain,
at least 30% of the ears of the male plants comprise pollen,
female plants receptive, wherein at least 30% of the ears of the female plants comprise gaping florets,
no substantial cloud coverage, and
after morning dew has evaporated and before temperatures in the field rise above 20° C.
